(19) United States
(12) Patent Application Publication
(10) Pub. No.: US 2025/0032054 A1
(43) Pub. Date: Jan. 30, 2025

(54) SYSTEM FOR CONCUSSION ASSESSMENT

(71) Applicant: VIRTUAL REALITY CONCUSSION ASSESSMENT CORPORATION, Raleigh, NC (US)

(72) Inventors: Shihching Yeh, Taichung City (TW); Eric Wu, Taipei City (TW); James M Poole, Raleigh, NC (US)

(21) Appl. No.: 18/915,584

(22) Filed: Oct. 15, 2024

Related U.S. Application Data
(63) Continuation of application No. 18/541,217, filed on Dec. 15, 2023.
Application No. 18/541,217 is a continuation-in-part of application No. 17/353,793, filed on Jun. 21, 2021, now abandoned.

(51) Int. Cl. A61B 5/00; A61B 5/16; A61B 5/369

(52) U.S. Cl. CPC A61B 5/7282; A61B 5/163; A61B 5/369; A61B 5/6898; A61B 5/7264; A61B 5/742; A61B 2560/0431

(57) ABSTRACT

The present invention relates to an improved device and method for the determination of a concussive state, while taking an EEG reading from the Fcz, Fc3 and Pz positions on the human and combining it with simultaneous eye tracking a more accurate measurement for determination of a concussive state can be had.

This application is a continuation of U.S. non-provisional application Ser. No. 18/541,217 filed on Dec. 15, 2023, which is a continuation-in-part of U.S. non-provisional application Ser. No. 17/353,793 filed on Jun. 21, 2021 and which is incorporated herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a system of assessment of the presence of a concussion. In particular, it relates to an improved system for concussion assessment using a combination of EEG and eye movement evaluation.

### Description of Related Art

The occurrence of mild traumatic brain injuries (mTBI) such as a concussion is frequently occurring in people that strenuously exercise. Activities such as basketball, football, soccer, boxing, MMA fighting, soldier actions, and the like all have a higher likelihood of concussion than other forms of activity. In the USA alone there are over 1 million mTBI cases a year.

Concussions can produce a disturbance of brain function in addition to possible structural injury, though there may be no obvious structural injury. People affected with mTBI may often exhibit no immediate visible post trauma abnormalities. Persons affected by mTBI may result in mechanical injury to the axonal, which can result in acute long-term damage and axonal degeneration. While there are several methods used to try and determine if a concussion has occurred, they all have their own drawbacks and a level of accuracy that needs improvement. It is therefore difficult to rely on a concussive determination with these methods.

### BRIEF SUMMARY OF THE INVENTION

The present invention relates to an improved device and method for the determination of a concussive event having occurred. It has been discovered that by taking an EEG with the electrodes on the Fcz, Fc3 and Pz position on the patient, when viewed together (not independent of one another) with eye tracking that the combination is a much more effective and accurate device and method of determining if a concussion has occurred.

Accordingly, in one embodiment, there is a system for determining a concussive state in a human comprising:
a) an electroencephalogram (EEG) for acquiring EEG readings from 3 electrodes positioned on the head of the human wherein the electrodes are placed at the Fcz, Fc3 and Pz positions on the human; and
b) an eye tracking device which consists of the ability to track eyes wherein what is measured consists of openness, visual axis of the eye and eye focus of the human while the EEG readings are being acquired.

In another embodiment, there is a method for a concussion assessment on a human comprising
a) obtaining an EEG with electrodes on the human Fcz, Fc3 and Pz positions on the human;
b) simultaneously with the EEG obtaining eye tracking of the human; and
c) sending the EEG and the obtained eye tracking to an analytical device for concussion assessment.

### BRIEF DESCRIPTION OF THE DRAWINGS

FIG. 1 is a map of the positions on the human head where EEG readings are taken.

FIGS. 2a, 2b, 3a, and 3b all show screen shots of videos that are used to get an eye tracking result.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publication may set out definitions of a term that conflict with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “concussive state” refers to mild mTBI in an individual. The present invention is designed to determine the presence of internal brain injury even if no outward physical symptoms are observed. Concussion can affect memory, judgment, reflexes, speech, balance, and muscle coordination. People with concussions often report a brief period of amnesia or forgetfulness, where they cannot remember what happened immediately before or after the injury. There is a danger if the mTBI is not diagnosed and treated, there may be permanent damage to an individual.

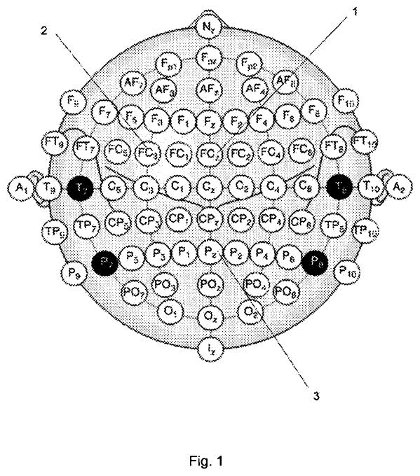

As used herein, the term “EEG” refers to an electroencephalogram (EEG), a test that measures electrical activity in the brain using small, metal discs (electrodes) attached to the scalp. Brain cells communicate via electrical impulses and are active all the time, even during asleep. This activity shows up as wavy lines on an EEG recording. There are as many as 50 sites on a patient's head (see FIG. 1) which EEG electrodes can be attached. Frequently 10 to 20 of these locations are utilized in an EEG. The electrodes of the present invention are placed at 3 specific locations, Fcz, Fc3 and Pz. It has been surprisingly discovered that only using these 3 locations gives a more accurate indication of a concussion when combined simultaneously with eye tracking.

As used herein, the term “electrodes” refers to the electrodes placed on the head during an EEG that are connected to the EEG. In the present invention only 3 electrodes are utilized, positioned on the Fcz, Fc3 and Pz locations on a patient's head.

As used herein, the term “Fcz, Fc3 and Pz positions on the human brain” refers to 3 medically recognized positions on the head of an individual. The location of these three sites can be seen in the FIG. 1 map and is where the 3 electrodes of the EEG device are positioned. The input from these 3 sites is combined with eye tracking done at the same time to give a concussive state assessment.

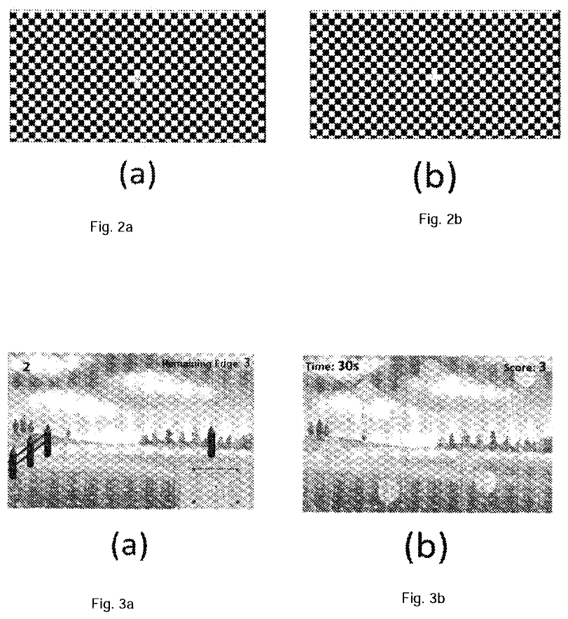

As used herein, the term “eye tracking device” refers to a device which measures certain eye parameters in response to one or more test images or videos shown to the patient. (some test images are seen in FIG. 2 and FIG. 3). The test images (still or motion) stimulate the patient's eyes and the movement during that time is combined with the EEG results to determine a concussive state. The eye tracking parameters that are measured are eye openness (blinking), change in the visual axis of the eye, and eye focus. In one embodiment the test image is presented to the patient via a head mounted display.

As used herein, the term “analytical device” refers to a computer or the like which can take (wired or wireless) the EEG results and the eye tracking results and do a concussive assessment of the presence of a concussive state. This can be on a computer, or a smart phone and it also can be driven by artificial intelligence. In one embodiment, the whole system is portable for use in the field like at the scene of an accident.

### DRAWINGS

Now referring to the drawings, FIG. 1 is a map of the top of the head of an individual showing the many locations that are used to place electrodes. The 3 of the present invention are Fcz 1, Fc3 2 and Pz 3 are all that's used. placement of the EEG electrodes is done by routine methods.

FIGS. 2a and 2b and FIGS. 3a and 3b are examples of animations that can be utilized to get an eye tracking result,

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. A system for determining a concussive state in a human comprising:
a) an electroencephalogram (EEG) for acquiring EEG readings from 3 electrodes positioned on the head of the human wherein the electrodes are placed at the Fcz, Fc3 and Pz positions on the human; and
b) an eye tracking device which consists of the ability to track eyes wherein what is measured consists of openness, visual axis of the eye and eye focus of the human while the EEG readings are being acquired.

2. The system according to claim 1 which further comprises an analytical device for analyzing the EEG reading and eye tracking together to assess if the human has a concussion.

3. The system according to claim 2 wherein the analytical device is driven by artificial intelligence machine learning.

4. The system according to claim 2 wherein the analytical device is a smart phone.

5. The system according to claim 1 wherein the eye tracking device consists of a head mounted display that displays at least one eye tracking and at least one EEG stimulating display.

6. The system according to claim 1 wherein the system is portable.

7. A method for a concussion assessment on a human comprising:
a) obtaining an EEG with electrodes on the human Fcz, Fc3 and Pz positions on the human;
b) simultaneously with the EEG obtaining eye tracking of the human; and
c) sending the EEG and the obtained eye tracking to an analytical device for concussion assessment.

8. The method for a concussion assessment on a human according to claim 7 wherein the analytical device is driven by artificial intelligence.

9. The method for a concussion assessment on a human according to claim 7 wherein the analytical device for concussion assessment is a smart phone.
